(19) United States
(12) Patent Application Publication
(10) Pub. No.: US 2005/0192649 A1
(43) Pub. Date: Sep. 1, 2005

(54) Systems and methods for providing variable medical information

(75) Inventors: Firass Shehadeh, Maple Grove, MN (US); James A. Esler, Coon Rapids, MN (US); Richard Fears, Moundsview, MN (US); Timothy R. H. Pratt, Arden Hills, MN (US)

Correspondence Address: JASON R. KRAUS;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 10/789,778

(22) Filed: Feb. 27, 2004

(51) Int. Cl. A61N 1/18

(52) U.S. Cl. 607/60

(57) ABSTRACT

Systems and methods for providing variable medical information. In some cases, the systems include elements operable to receive information from a number of implantable medical devices and to provide a common format information set. Thus, in one exemplary case, two types of encoded binary information is received from different implantable medical devices. This information can be converted to a desired format, and assembled into a common medical information database.

### CROSS-REFERENCE TO RELATED APPLICATIONS

The present application is related to six co-pending patent applications: U.S. patent application No. ______ entitled “SYSTEMS AND METHODS FOR ACCESSING AND DISTRIBUTING MEDICAL INFORMATION” and filed by Jones et al. (Attorney Docket No. 300564); U.S. patent application No. ______ entitled “SYSTEMS AND METHODS FOR DELIVERING AND GATHERING MEDICAL DIAGNOSTIC DATA” and filed by Rossinni et al. (Attorney Docket No. 300566); U.S. patent application No. ______ entitled “SYSTEMS AND METHODS FOR AUTOMATICALLY COLLECTING, FORMATTING AND STORING MEDICAL DEVICE DATA IN A DATABASE” and filed by Esler et al. (Attorney Docket No. 300567); U.S. patent application No. ______ entitled “SYSTEMS AND METHODS FOR UPLOADING AND DISTRIBUTING MEDICAL DATA SETS” and filed by Fears et al. (Attorney Docket No. 300568); U.S. patemt application No. ______ entitled “SYSTEMS AND METHODS FOR VALIDATING PATIENT AND MEDICAL DEVICE INFORMATION” and filed by Pratt et al.(Attorney Docket No. 300569); and U.S. patemt application No. ______ entitled “SYSTEMS AND METHODS FOR AUTHORIZING AND PROCESSING REIMBURSEMENTS FOR SERVICES PROVIDED IN THE COLLECTION OF IMPLANTABLE MEDICAL DEVICE DATA” and filed by Stawski et al. (Attorney Docket No. 301131). Each of the above-identified applications is filed on a date even herewith, and each of the above-identified applications is hereby incorporated by reference for all purposes.

### BACKGROUND OF THE INVENTION

The present invention is related to implantable medical devices, and in particular to systems and methods for accessing information derived from disparate implantable medical devices.

In a typical scenario a patient is implanted with a medical device that provides a desired therapy. At various intervals, the implanted medical device is queried by a programmer located at the office of the patient's physician. The information obtained from the implanted medical device is then stored on a patient disk. Further, various of the information can be printed out and maintained in a patient's file. It is, however, difficult to access a patient's full history for comparison reasons.

Hence, for at least the aforementioned reason, advanced systems and methods are desirable.

### BRIEF SUMMARY OF THE INVENTION

The present invention provides systems and methods for accessing information derived from disparate implantable medical devices. In one exemplary embodiment of the present invention, a system is provided that is capable of receiving information derived from a plurality of implantable medical device types and for converting and/or abstracting the information for presentation in a common format. Thus, for example information can be derived from one implantable medical device in a compressed format, and from another implantable medical device in a binary format. All of this information can be modified to a desired end format such as, for example, an XML format using field tags common to both information sets.

Some embodiments of the present invention provide systems for translating medical data. Such systems include two or more interpretation systems that are operable to receive encoded data sets from respective implantable medical device types, and to create a decoded data set therefrom. Further, a plurality of abstraction engines are provided that are each operable to receive the respective decoded data sets, and to provide abstracted data sets therefrom. These abstracted data sets are provided in a format common to various implantable medical device types. In some cases, the abstracted data sets are stored to a comprehensive data base, while in other cases, at least a portion of the abstracted data sets are distributed to one or more recipients. These recipients can be network servers and/or various subset databases.

In some instances, the systems further include a communication link corresponding to each of the interpretation systems. These communication links are operable to receive the encoded data sets originating from implantable medical devices. In some cases, the communication links are server ports. These server ports can be assigned to particular medical device types, and thus data received via a particular port is known to be data from a particular medical device type.

A server associated with the server ports can include a processor as well as a computer readable medium. The computer readable medium includes instructions executable by the processor to receive an encoded data set from one of a plurality of implantable medical device types; identify which of the medical device types originated the encoded data set; and communicate the encoded data set via a communication link associated with the identified medical device type to an interpretation system associated with the communication link.

Other embodiments of the present invention provide methods for utilizing information from implantable medical devices. Such methods include providing two or more communication links each associated with respective conversion utilities, and each associated with particular medical device types. A data set is received from a medical device and is communicated via a communication link associated with that medical device. The information is converted to a converted data set using a conversion utility associated with the communication link via which the information is received.

Yet other embodiments of the present invention provide systems for translating medical data that include a data translation system with a processor and a computer readable medium. The computer readable medium includes instructions executable by the processor to: receive an encoded data set from one of a plurality of implantable medical device types via one of a plurality of ports where the ports are assigned to different types of implantable medical devices; select a conversion utility based at least in part on the port via which the encoded data set is received from; spawn the conversion utility; and translate the encoded data set to a decoded data set.

In some cases, another processor and computer readable medium is provided. The other computer readable medium includes instructions executable by the other processor to: receive the encoded data set from a particular implantable medical device; identify the one of the plurality of medical device types; and direct the encoded data set to the one of the plurality of ports corresponding to the one of the plurality of implantable medical device types.

This summary provides only a general outline of some embodiments according to the present invention. Many other objects, features, advantages and other embodiments of the present invention will become more fully apparent from the following detailed description, the appended claims and the accompanying drawings.

### BRIEF DESCRIPTION OF THE DRAWINGS

In the Figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 depicts a prior art system for gathering medical information;

FIG. 2 is a schematic drawing of one embodiment of a system for gathering, validating, storing, using and/or distributing medical device information;

FIG. 3 is a flow diagram illustrating a method for uploading medical data in accordance with some embodiments of the present invention;

FIG. 4 is a block diagram of a translation system in accordance with various embodiments of the present invention;

FIG. 5 is a flow diagram illustrating a method in accordance with the present invention for operating the translation system of FIG. 4;

FIG. 6a is a flow diagram illustrating a method for performing clinical and/or operational trials in accordance with some embodiments of the present invention;

FIG. 6b is a flow diagram illustrating one embodiment of a method for performing the “validate participant-entered information” task of the method of FIG. 6a;

FIG. 6c is a flow diagram illustrating one embodiment of a method for performing the “validate device data” task of the method of FIG. 6a;

FIG. 6d is a flow diagram illustrating one embodiment of a method for performing the “process participant payment” task of the method of FIG. 6a;

FIG. 6e is a flow diagram illustrating one embodiment of a method for performing the “populate databases” task of the method of FIG. 6a;

FIG. 7 is a flow diagram illustrating a method for obtaining analysis associated with one or more medical data sets in accordance with some embodiments of the present invention;

FIG. 8 is an exemplary graphical analysis request interface in accordance with various embodiments of the present invention; and

FIG. 9 is a flow diagram illustrating a method for providing a diagnosis or other analysis based on a medical data set in accordance with some embodiments of the present invention.

### DETAILED DESCRIPTION OF THE INVENTION

The present invention provides systems and methods for gathering and/or distributing medical information. In one exemplary embodiment, the present invention provides for medical information gathered from one or more patients to be maintained on a central database that is subsequently accessible to the patient, the patient's physician, researchers, and/or other interested parties. Thus, for example, a patient may visit their physician and during that visit the physician may read information from an implantable medical device associated with the patient, make other objective measurements of the patient, and record various subjective information about the patient. All of this information can be uploaded and maintained on a variably accessible system. The system is variably accessible as access rights to the maintained medical information may be different for each interested party based on privacy, need and/or business concerns.

As used herein, the term “implantable medical device” is used in its broadest sense to mean any medical device that is either implanted within a living being, or integrally associated with a living being. As just one example, a pacemaker is an implantable medical device. Further, as used herein, the term “subjective information” is used in its broadest sense to mean information based on an interpretation of a human being. Thus, for example, a physician may indicate that a particular person appears “happy” and/or “healthy”. Both of these determinations are considered subjective information. Alternatively, as used herein, the term “objective information” is used in its broadest sense to mean any information based on an objective measurement. Thus, for example, a physician may indicate that a patient is a certain weight or has a certain blood pressure based on measurements. These are both examples of objective data. In addition, certain diagnosis or patient history information might be a combination of subjective and objective information. For example, the fact that a patient has a history of coronary artery disease, or some other disease probably is objective information based on a subjective diagnosis from the past.

In some cases, systems and methods of the present invention can be used to perform clinical trials of medical devices. In such cases, information can be garnered from physicians overseeing patients utilizing a class of medical devices undergoing trial. The physicians can use programmers to read the medical devices, and the information derived from the medical devices can be uploaded via a communication network to a raw database. As used herein, the term “raw database” implies a computer readable medium that includes information in a less than finally converted format. Thus, as just one example, a raw database may include information received from an implantable medical device, or some derivative of such information that is not yet in an ultimately desired format.

The systems and methods can further include one or both of objective and subjective information about a patient. This information can be uploaded to the system via a communication network. As use herein, the term “communication network” is used in its broadest sense to mean any network or medium capable of passing information. Thus, a communication network can be, but is not limited to, the Internet, a cellular telephone network, a public switched telephone network, a local area network, a wide area network, a virtual private network, and/or combinations thereof.

In some cases, physicians, patients and/or other health care personnel are paid for gathering various medical data. In such cases, once the information is received by the system and validated, an agreed upon payment for gathering the information can be approved and disbursed to the physician, patient and/or other medical personnel. The gathered medical data can include subjective data collected by a physician, objective data collected by a physician, and/or a data set received from the implantable medical device.

Various embodiments of the present invention provide general purpose medical data access systems. Such systems include a system controller communicably coupled to a gateway controller. As used herein, a gateway controller can be any ingress or egress point where information comes into or leaves the system. Further, as used herein, a system controller can be any point where disparate portions of medical data are combined and/or converted. Thus, in some cases, a system controller and a gateway controller can be the same type of device and/or serve overlapping purposes. In one particular case, a gateway controller and a system controller are both servers communicably coupled to a communication network.

The gateway controller is operable to receive information from one or more sources of medical information, and in some cases to distribute various types of medical information. In one case, the gateway controller includes a processor and a computer readable medium. The computer readable medium includes instructions executable by the processor to receive data sets comprising objective and/or subjective data collected by a physician, and to communicate at least a portion of these data sets to the system controller. In one particular case, both the gateway controller and the system controller are implemented as a single computer system.

The system controller is operable to maintain one or more pieces of medical information, to translate various pieces of medical information to a desired format, and in some cases to distribute various portions of medical information to one or more recipient systems. The system controller includes a processor and a computer readable medium. The computer readable medium includes instructions executable to receive information from one or more sources including, but not limited to, a data set in a first format from an implantable medical device. The instructions are further executable to store the data set from the implantable medical device to a raw database, and to identify a group associated with the implantable medical device and based on the group, to select an interpreter. The interpreter is applied to the received data set such that the data set is converted from the first format to a second format. At least a portion of the data set converted to the second format is stored to a database associated with the gateway controller. Instructions are also included to validate the objective and subjective data collected by the physician.

In some cases, the systems further include a diagnostic controller (e.g., another gateway controller associated with a diagnostic system) communicably coupled to the system controller. The diagnostic controller is operable to provide various medical information to one or more recipients. This medical information can be diagnostic limited information that can be shared without implicating privacy concerns. As used herein, the term “diagnostic limited information” includes any information relied upon by a physician or other medically trained personnel to provide a medical diagnosis. In some cases, but not necessarily all cases, diagnostic limited information is stripped of all information that would lend itself to identifying the patient that the information describes.

In particular cases, various medical data is provided to one or more recipients who, in turn, opine upon the meaning of the provided medical information by providing an analysis or diagnosis based on the received medical information. This opinion information is received as analysis data (e.g., a medical diagnosis) at the diagnostic controller. In some cases, this analysis data is stored by the diagnostic controller in relation to the corresponding medical data, while in other cases, this analysis information is provided to the system controller where it is stored in a comprehensive database relative to the corresponding medical data. Thus, as just one example, a group of ten physicians may receive an electrocardiogram from a particular patient. In turn, each of the ten physicians opine as to what type of arrhythmia is indicated by the electrocardiogram, and the opinions are stored in relation to the electrocardiogram.

In particular cases, the diagnostic controller can further operate to provide diagnosis guidance to one or more system users. For example, a diagnosis query including “specific diagnostic limited data” may be received at the system. As used herein, the term “specific diagnostic limited data” includes a medical data set submitted for diagnostic purposes. In some cases, but not necessarily all cases, specific diagnostic limited data is stripped of information that would lend itself to identifying the patient that the information describes. The received specific diagnostic limited data is compared to at least a portion of the diagnostic limited information and a closest match is determined. Based at least in part on this closest match, a diagnosis is provided. Thus, as just one example, a physician can submit an electrocardiogram associated with a patient. A database is queried to determine whether relevant matches between the presented electrocardiogram and other previously analyzed electrocardiograms exist. Where one or more close matches are found, the diagnosis associated with the matched electrocardiograms is/are provided to the requester.

In various embodiments of the present invention, information from implantable medical devices is received at a system controller where it is stored in a first format to a raw database. At least some of the information maintained on the raw database is converted to a desired format and stored to a comprehensive database and/or distributed to one or more subset databases. As used herein, the term “comprehensive database” indicates a database where a superset of data is maintained, and the term “subset database” indicates a database where a subset (i.e., a portion of the superset) of the data is maintained. Such subset databases can be, for example, associated with controllers operable to perform different functions and the subset can be chosen based on the function to be performed. As just one of many examples, where access is to be given to a patient's physician, the subset database may include patient specific information including patient name and address information. As another of many examples, a subset database can be a diagnostic database used by one or more physicians, researchers, or the like.

In various cases, information can be accessed from the raw database, and from the accessed information, one or more subset databases can be created. Thus, for example, where a subset database is lost, it can be regenerated from the raw database. Alternatively, or in addition, where an original formation of a subset database is later found to be inadequate, the subset database can be created anew based on a different portion of the raw database, a different data conversion, and/or the like.

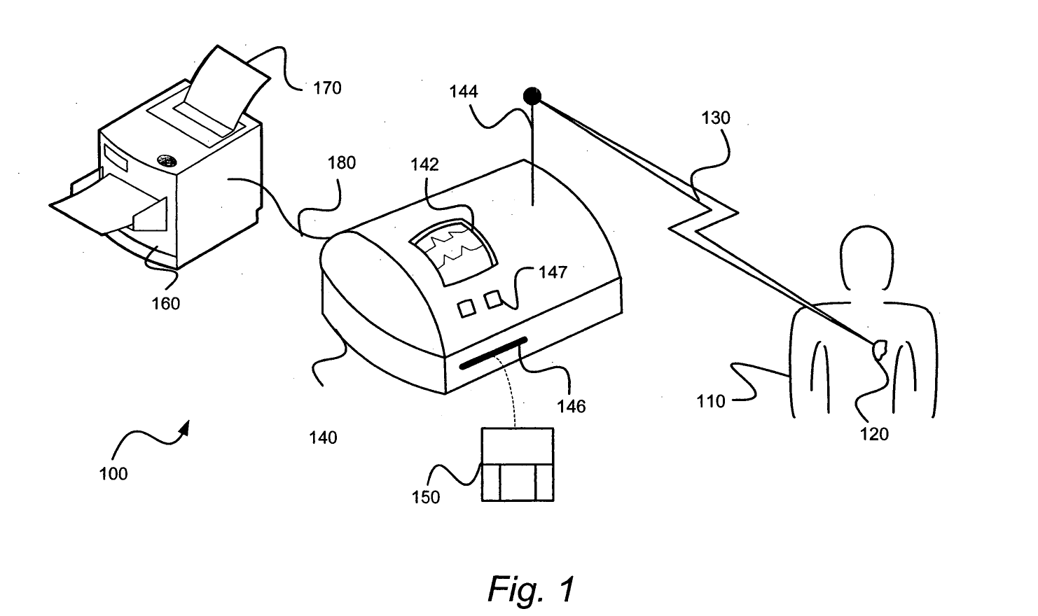

Turning to FIG. 1, a prior art system 100 for gathering medical information is depicted. System 100 includes a patient 110 having an implantable medical device 120. Patient 110 visits a physician's office that includes a programmer 140 capable of communicating with implantable medical device 120 via a communication link 130. Programmer 140 includes an antenna 144 to facilitate such communications. Programmer 140 includes an insertion bay 146 capable of receiving a removable computer readable medium 150 such as, for example, a floppy disk. In addition, programmer 140 includes a graphical display 142 and one or more user input devices 147 for controlling programmer 140.

Programmer 140 is electrically coupled, for example, via a wire 180 to a printer 160 capable of printing information on paper 170. As used herein, the term “electrically coupled” is used in its broadest sense and includes any coupling whereby electrons forming part of a communication can pass. Thus, for example, a wire physically attaching two devices can be considered to electrically couple the two devices. In contrast, as used herein, the term “communicably coupled” is broader than and encompasses electrically coupled. In particular, communicably coupled includes any coupling whereby information can be passed from a source to a destination. Thus, two devices can be communicably coupled where they communicate via a wire, and/or where they communicate wirelessly.

In operation, patient 110 visits the physician's office where the physician causes programmer 140 to query implantable medical device 120. Information is transferred from implantable medical device 120, which typically is in some encoded binary format, such as binary coded decimal (BCD) or the like. Further, the encoded binary information can include a combination of different formats, for example, some data being in BCD format and other data being in some proprietary format. This information is passed to an interpreter included as part of programmer 140, which decodes the information to a graphical format displayed via graphical display 142. For example, the information can include a group of Cartesian coordinate data that can be displayed as, for example, and electrocardiogram.

Further, it should be recognized that implantable medical device 120 can provide a number of parameters as part of the information uploaded to programmer 140. In some cases, implantable medical device 120 can provide seven hundred to fifteen hundred different parameters, or more. The number of parameters provided is specific to a given implantable medical device and/or programming mode and, thus, a given programmer typically is specific to one type of implantable medical devices and/or a group of implantable medical devices.

In addition to displaying graphical information derived from the information passed from implantable medical device 120, the physician can save the information from programmer 140 onto removable computer readable medium 150. Further, the physician can print the information in a graphical format on paper 170 for study and/or placement in the patient's file. However, by using such a system the physician cannot display a historical record electronically that covers multiple patient visits. This limits the physician's ability to function efficiently.

Further, in a clinical trial scenario, the physician sends removable computer readable medium 150, along with handwritten questionnaire to the manufacturer of implantable medical device 120. This information must then be transferred to a database and verified by a human. This is inefficient and/or error prone.

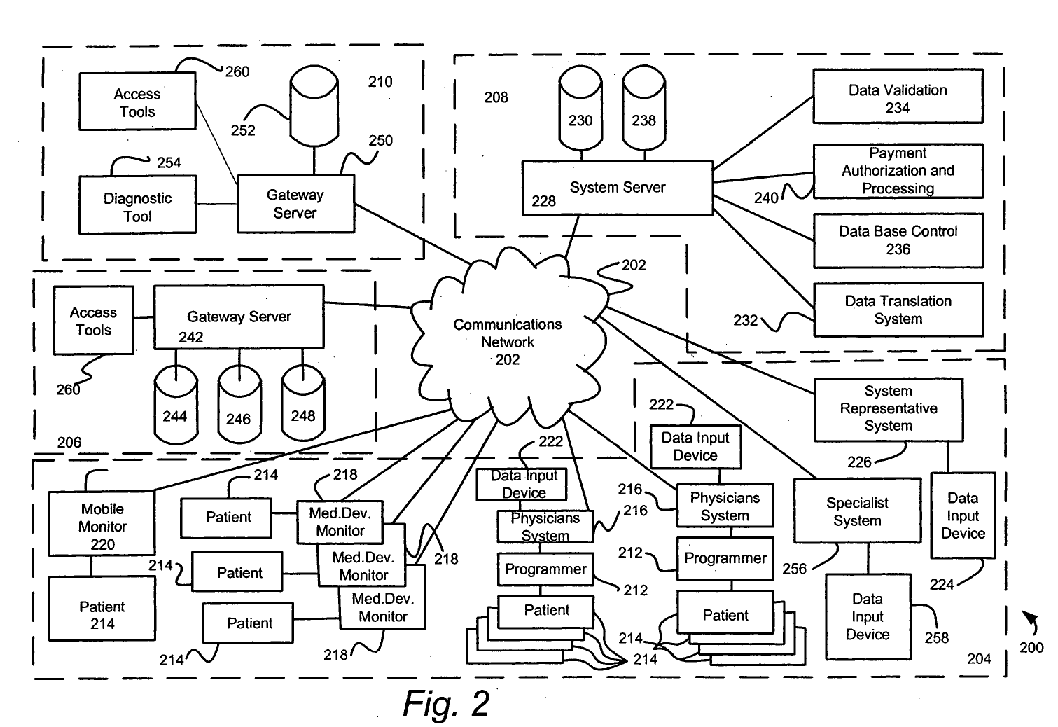

Turning now to FIG. 2, one embodiment of a system 200 for gathering, validating, storing, using, manipulating and/or distributing medical information is shown. In the illustrated embodiment, system 200 is configured to receive and maintain medical and other healthcare information on a central database repository system. System 200 also is configured to make some or all of the information accessible to medical device manufacturers, the patient, the patient's physician(s), and/or perhaps other third party healthcare providers, researchers, service companies, or organizations. Thus, as illustrated in FIG. 2, system 200 comprises a number of computer-based systems communicating via a communication network 202. In one embodiment, system 200 comprises data input devices 204, a data collection system 206, a central data processing and repository system 208, and a medical diagnostic system 210, all communicating with one or more of the other systems via communication network 202.

In accordance with one embodiment of the present invention, data input devices 204 are adapted to receive implantable medical device information, and to communicate that information to the various systems. U.S. patent application Ser. No. 10/422,495, filed on Apr. 23, 2003 by Bardy and entitled “System and Method for Providing Tiered Patient Feedback For Use in Automated Patient Care”, and U.S. Pat. No. 6,607,485, filed on Sep. 6, 2001 by Bardy and entitled “Computer Readable Storage Medium Containing Code for Automated Collection and Analysis of Patient Information Retrieved From an Implantable Medical Device For Remote Patient Care”, provide additional information about transferring information from an implantable medical device via a communication network. The entirety of each of U.S. Pat. No. 6,607,485 and U.S. patent application Ser. No. 10/422,495 is incorporated herein by reference for all purposes.

In addition to the information collected from the implantable medical devices, objective patient information, subjective patient information, and/or diagnosis information can be received and communicated to the various systems. Such additional information can be, but is not limited to, a quality of life measure describing the patient's physical and emotional well-being and a record of symptoms, such as provided by a Duke Activity Status Indicator™. Other types of measures can also be used including, for example, the Minnesota Living with Heart Failure Questionnaire described in E. Braunwald, ed., “Heart Disease—A Textbook of Cardiovascular Medicine,” pp. 452-454, W. B. Saunders Co. (1997), the disclosure of which is incorporated herein by reference for all purposes. As other examples, functional classification standards defining relationships between symptoms and the amount of effort required to provoke such symptoms, Such as the New York Heart Association classifications I, II, III and IV described in the aforementioned textbook can be provided to the system. Based on the disclosure provided herein, one of ordinary skill in the art will recognize a myriad of information in addition to implantable medical information that can be provided to system 200.

As an example, programmers 212 typically located in physicians' offices are adapted to receive information from medical devices implanted in patients 214. However, instead of dumping the data onto a diskette as disclosed above, programmers 212 are connected to communication network 212, and are configured to upload the medical device information to one or more of systems 206, 208 and 210. In one embodiment, programmers 212 are connected directly to communication network 202. In another embodiment, programmers 212 might be connected to communication network 202 through the physicians' information systems 216 or through other intermediary systems. As one skilled in the art will appreciate, however, the particular means by which programmers 212 are connected to communication network 202 are not important, so long as programmers 212 can communicate the medical device information to network 202 and systems 206, 208, and 210.

In accordance with another embodiment of the invention, patients 214 have medical device monitors 218 located at their residences, which are configured to receive and transmit medical device information to communication network 202. In accordance with this embodiment, like programmers 212, monitors 218 receive information from the medical devices implanted in patients 214 via a wireless communication connection. In one embodiment, monitors 218 can be configured to retrieve the medical device information on a periodic basis; for example, every night while the patient sleeps or perhaps weekly. Alternatively, monitors 218 can be configured so that the patient dictates when the data transfer occurs, or monitors 218 can be configured to perform a data transfer when a triggering episode (e.g., an abnormal event) is detected.

After monitors 218 receive the information from the implanted medical devices, monitors 218 then upload the information to communication network 202 via a communication connection. As one skilled in the art will appreciate, the communication connection to network 202 can be any suitable connection, such as an Internet connection, a wired telephone connection, a wire connection, or any other suitable communication connection currently known or hereinafter developed. Further, it will be recognized that communication network 202 can be any network capable of facilitating communications including, but not limited to, the Internet, a cellular telephone network, a public switched telephone network, a local area network, a wide area network, a virtual private network, or combinations thereof.

In accordance with yet another embodiment of the invention, the implanted medical device information can be uploaded to communication network 202 from a mobile device monitor 220. Such a mobile device monitor 220 can be integrated with a cellular telephone. In accordance with this embodiment, mobile device monitor 220 is similar to monitor 218 except that patient can carry monitor 220 with him/her whenever necessary. Because device monitor 220 is mobile, its connection to communication network 202 most likely will be a wireless connection; however, the present invention is not limited to this embodiment. One skilled in the art will appreciate that mobile device monitor can communicate with network 202 via any suitable communication connection.

As mentioned above, in addition to medical device information, objective patient information, subjective patient information, diagnostic information, as well as other medical information can be input and uploaded to systems 206, 208 and 210. As discussed above, “objective information” means any information based on an objective measurement, such as a patient's weight, blood pressure measurements, etc. Further, “subjective information” means information based on an interpretation of a human being, such as a diagnosis of a patient's medical condition or symptoms. In addition, certain diagnosis or patient history information might be a combination of subjective and objective information. For example, the fact that a patient has a history of coronary artery disease, or some other disease probably is objective information based on a subjective diagnosis from the past. Regardless of the classification of the information, the present invention can be configured to collect, store and use any suitable medical, diagnostic, or other patient information one deems appropriate. Thus, the present invention is not limited to any particular type or form of information collected.

In one embodiment of the present invention, physicians can use data input devices 220 to enter patient information, including objective and subjective information. Further, data input devices 220 can be used to verify medical information and/or provide analysis input corresponding to medical information. Data input devices 220 can be any suitable data input device, such as, a personal computer, a mobile computing device, or a cellular or wireless device. In one exemplary embodiment, data input devices 220 are personal digital assistants (PDA) with integrated wireless communication capability. In addition, the data can be entered using any number of different software programs. For example, data input device 220 can include a data entry questionnaire program, which prompts the physician to enter specific information. Alternatively, data input device 220 may include a web browser for processing a data entry web page or applet. As one skilled in the art will appreciate, any suitable device and/or method can be used to enter the patient information, and thus, the present invention is not limited to any particular embodiment or configuration.

In the illustrated embodiment, data input device 220 is connected to communication network 202 through physician's information system 216. Alternatively, data input device 220 can be connected directly to communication network 202 or can be connected through some other intermediary system. As one skilled in the art will appreciate, however, the particular means by which data input device 222 is connected to communication network 202 is not important, so long as the device can communicate the medical, diagnostic and/or other patient information to network 202 and systems 206, 208, and/or 210.

In some instances, a physician may not have access to a system for entering patient information. In those situations, the physician may record the medical, diagnostic and other patient information on charts or forms, or the physician may dictate the information onto an audio recordable medium. When this occurs, the physician may send the charts, forms, and/or audio recordable medium to a data input representative. In this instance, the representative will enter the patient information (including objective and subjective information) using a data input system (not shown).

In some cases, a physician entering information into system 200 may be required to verify and/or authenticate the information after acceptance into system 200. In such a case, the physician may request to view previously entered data via data input device 222. In turn, the physician may verify the data and provide an indication of the data validity and/or authenticity via data input device 222. Where the data is not available, the physician may send a communication via data input device 222 to a system representative system 226. A system representative utilizing data input device 224 can determine the availability of the requested data, and in real time provide the data to data input device 222 where the physician can verify and/or authenticate the data.

As discussed above, implantable medical device information can be input and uploaded using a number of different devices, including programmers 212, medical device monitors 218, and mobile monitors 220. After programmers 212 and/or monitors 218, 220 have received the medical device information, they upload the data to central data processing and repository system 208 via communication network 202. In accordance with this aspect of the invention, central data processing and repository system 208 comprises a server 226 for receiving the medical device information and storing it on a raw medical device data database 228.

As one skilled in the art will appreciate, data from implantable medical devices typically is in an encoded binary format. In one embodiment, programmers 212, and medical device monitors 218, 220 can be configured to decode the medical device data streams prior to uploading them to system server 228 and raw medical device data database (“raw database”) 230. In accordance with this embodiment, the uploaded data stream is decoded, but still is in binary format. In an alternative embodiment, programmers 212 and medical device monitors 218, 220 merely receive and upload the medical device data without performing any decoding function. In this embodiment, central data processing and repository system 208 will perform the decoding function. In either case, however, raw database 230 stores the medical device information in a binary format, which makes it difficult for many programs and databases to use.

Thus, in one embodiment, it is desirable to convert the medical device data in binary format to a more database friendly format. In accordance with this aspect of the invention, a data translation module or system 232 receives the medical device data in binary format and converts the data to a more “user-friendly” format, such as the extensible mark-up language (“XML”) format. As discussed in more detail below, data translation system 232 parses the binary data and moves at least some of the data into XML fields.

After the data is formatted into, for example, XML fields, the XML data then is stored in a data warehouse. In one embodiment, the XML data may be stored in comprehensive database 238, or some other database location. Next, a data validation module or system 234 receives the XML data and validates the accuracy of the data translation. Data validation system 234 can perform other functions, as well, such as remove redundant XML data and validate and/or normalize physician-entered information. The operation of data validation system 234 is discussed in more detail below.

After the XML data has been validated, it then can be populated into a database for use by other programs. In accordance with this aspect of the invention, a database control module or system 236 reads the XML data and populates it into a predefined database such as, for example comprehensive database 238 and/or subset databases 248, 252. Once populated, all or part of database 238 can be made available to a number of different entities for querying and use. The operation of database control system 236 will be discussed in more detail below.

As one skilled in the art will appreciate, when a physician participates in a medical device study, the physician typically is compensated by the entity running the study for the physician's time and effort. For example, a medical device manufacturer might procure a study to verify certain functionality or to gain FDA approval for the device. In that situation, the medical device manufacturer will enlist the help of physicians to take part in the study, which includes implanting medical devices in patients, conducting follow-up evaluations of the patients and performance of the medical devices, collecting data from the implanted medical devices, and providing the data to the medical device manufacturer for analysis. Typically, after a physician performs these functions, the entity running the study will verify that the physician performed the functions correctly, and then will reimburse the physician. In accordance with this aspect of the invention, a reimbursement authorization and processing module or system 240 will verify or validate that the physician followed the medical device study parameters properly, and upon validation will interface with an accounting system so the physician is reimbursed. The operation of reimbursement authorization and processing system 240 will be discussed in more detail below.

In the illustrated embodiment, data translation system 232, data validation system 234, database control system 236, and reimbursement authorization and processing system 240 are all illustrated as separate systems within data processing and repository system 208. These systems, however, are illustrated as separate systems merely to describe the functionality of the modules. One skilled in the art will appreciate that the functionality of these systems or modules can be incorporated into a single processing system, such as system server 228, and thus, the present invention is not limited to the distributed embodiment illustrated in FIG. 2. In addition, while the operation of data translation system 232, data validation system 234, database control system 236, and reimbursement authorization and processing system 240 are described separately below, one skilled in the art will appreciate that the functionality performed in each system or module is not limited necessarily to module in which it is described. Some of the functionality of the modules could possibly be performed by another module. For example, while data validation and database control are described as separate modules, they possible could be combined as a single module, or perhaps, some but not all validation might be performed by database control system 236.

As mentioned above, in addition to medical device information, objective patient information, subjective patient information, diagnostic information, as well as other medical information can be input and uploaded to various systems via communication network 202. In accordance with one embodiment of the invention, data collection system 206 is configured to receive the objective and subjective patient information from the physician systems 216 or from representative system 226. In one embodiment, data collection system 206 includes a gateway server 242 for receiving the objective and subjective data from communication network 202, and one or more databases for storing the data. In the illustrated embodiment, data collection system 206 comprises an objective database 244, a subjective database 246 and a subset database 248. Objective database 244 stores the “objective information” entered by a physician, and subjective database 246 stores the “subjective information” entered the physician. While these databases are shown as separate database, it is for illustration purposes only. One skilled in the art will appreciate that data collection system 206 could be, and in many instances, probably will be configured with only a single database for both objective and subjective information.

After data collection system 206 obtains the objective and subjective data from the physicians, it uploads the data to data processing and repository system 208, where it is validated and loaded into comprehensive database 238 along with the implantable medical device data. Once populated, comprehensive database 238 will include medical device information and subjective and objective information for each patient. As discussed above, medical device information can be collected during patient visits to the doctor, or it can be collected at predefined intervals at the patients home or other locations using medical device monitors 218 or mobile monitors 220. The objective and/or subjective, however, typically will only be entered by a physician after the physician analyses and/or diagnosis the patient during a patient visit.

After the data is collected in comprehensive database 238, some or all of the data can be made available to different entities or third parties. For example, a portion of database 238 can be downloaded to data collection system 206 and stored in subset database 248. Then, physicians, patients, or other third party healthcare providers can access this smaller subset of data from data collection system 206, for example, by querying subset database 248 through communication network 202 and gateway server 242. As one skilled in the art will appreciate, third parties can access subset database 248 a number of different ways, including, for example, using a database structured query language, using software programs adapted to access the database, or using web pages or applet having embedded database calls.

In the embodiment illustrated in FIG. 2, data collection system 206 and data processing and repository system 208 are shown as separate systems. In one embodiment, the systems are separate as illustrated. For example, in one embodiment, data processing and repository system 208 might be a system located at and maintained by a medical device manufacturer, and data collection system 206 might be third party data collection service or agency. In this embodiment, data processing and repository system 208 might be configured to collect the medical device data, and data collection system 206 might be configured to collect objective and subjective data from the physicians, as discussed above.

Alternatively, in another embodiment, data collection system 206 could be configured to collect the medical device data and the objective and subjective data, and then upload all the information to data processing and repository system 208 for decoding, translation, validation, and/or database control and loading. In yet another embodiment, data collection system 206 could include one or more of data translation system 232 and data validation system 234. In still another embodiment, data collection system 206 and data processing and repository system 208 could be configured as a single system at one location, or a single system located at multiple sites. Because the systems are networked systems, any networked system configuration could be used.

The collected medical device and patient information can be used for a number of different purposes. In one embodiment, the collected data can be used to obtain FDA approval for a device, or the data can be used to publish a paper about a particular device and how to use the device. In accordance with another embodiment, the collected data is used to obtain and perhaps distribute diagnostic and other analysis information. In accordance with this embodiment, medical diagnostic system 210 can be configured to solicit diagnostic information from physicians and/or specialists, and after collecting the diagnostic information, distributing diagnoses to physicians or other third party healthcare providers based on entered symptom information.

In the illustrated embodiment, medical diagnostic system 210 receives at least a portion of the data from comprehensive database 238 and stores it in subset database 252. In one embodiment, medical diagnostic system 210 includes a gateway server 250 for receiving and storing the data in database 252. Medical diagnostic system 210 receives at least enough data for a physician or specialist to formulate a medical diagnosis. For example, the data may include information about a patient's symptoms, medical device readings, and any other suitable data that a physician may need to render a diagnosis. One skilled in the art will appreciate, the types and amount of data needed to render a diagnosis will depend on a number of factors, including the type of medical condition and medical device at issue.

In accordance with one embodiment, medical diagnostic system 210 also includes a diagnostic tool system or module 254 that is adapted to package medical data and deliver it to physicians and/or specialist in order to obtain a diagnosis from them. As discussed in more detail below, diagnostic tool 254 can be configured to package the medical data into a viewable package, such as a graphical web page or web applet, an email with readable attachments, or some other form of data communication.

After the medical data package is formatted, diagnostic tool 254 then sends the package to a specialist system 256 or a physician system 216. In the illustrated embodiment, specialist system 256 has a data input device 258 in communication with it, which is configured so that a specialist can review the medical data package and input diagnosis information based on his/her review of the medical data. Similarly, physician system 216 also can have a data input device 222 in communication with it for entering diagnosis information. Data input devices 258 and 222 can be any suitable input device, such as a personal computer, a handheld computing device, a cellular device, or the like.

After the specialists and/or the physicians enter the diagnosis information, it is sent back to medical diagnostic system 210 and saved in subset database 252, where it can be analyzed and processed. For example, in one embodiment, diagnostic tool module 254 may be configured to analyze diagnostic information provided by a number of different specialists, physicians, or other healthcare providers, and develop diagnoses and therapy suggestions based on input parameters. For example, a physician can enter patient symptoms and medical device information into a program or web page, and diagnostic tool 254 can be configured to determine and provide a diagnosis and perhaps a therapy suggestion by comparing the entered symptoms with medical data and diagnoses information stored in database 252. In this manner, the diagnostic tool can operate as an intelligent diagnostic machine. As with data collection system 206, medical diagnostic system 208 can be a stand-alone system operated separately from data processing and repository system 208, or it can be integrated with systems 208 and 206. The particular network configuration is not important.

In the illustrated embodiment, data collection system 206 and medical diagnostic system 210 both include access tools 260. As discussed in more detail below, access tools 260 are a set of interface tools, security devices, and access rules that allow users to have secure and perhaps limited access to the systems.

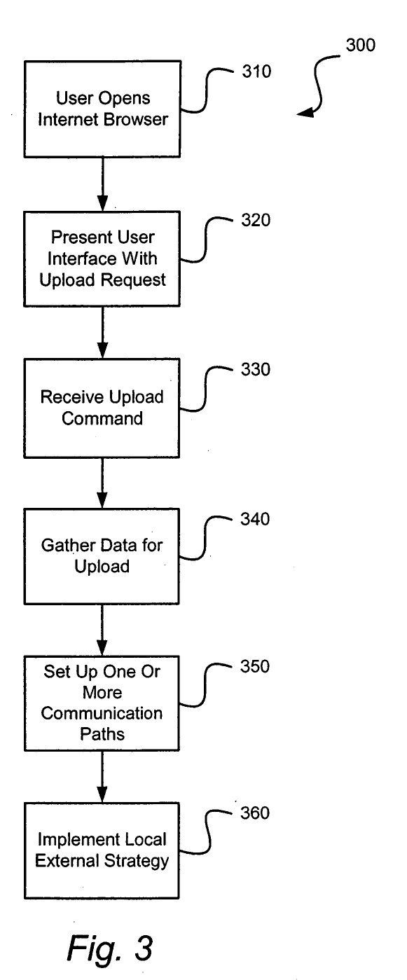

Turning now to FIG. 3, a user opens an Internet browser or some other communication tool installed on a microprocessor based device local to the user (block 310). The Internet browser can be installed on, for example, a mobile monitor, a bedside monitor, a mobile input device, a personal computer, a programmer, and/or the like. When the Internet browser is opened, the user can be automatically directed to a server that is operable to receive uploaded information, or a proxy thereof. In other cases, the user may have to direct the Internet browser to the appropriate site.

In some cases, a user first downloads an applet via a communication network that is executed locally. Such an applet can be downloaded from, for example, access tools 260 associated with data collection system 206. In one particular case, the applet is written in JAVA code, and may use a special plug-in to operate properly depending on the particular browser chosen. Other approaches for preparing to upload data as are known in the art may also be used.

In some cases, where an applet is downloaded, the user is presented with a dialog box requesting various authentication and/or authorization information as is known in the art. Once the authentication and/or authorization information is received, the applet is enabled to upload data to the system. Further, in some cases, the applet comes with an authentication certificate requesting that the user indicate the applet was received from a known and/or trusted source. In some cases, authentication and/or authorization is required each time information is to be uploaded to the system.

The Internet browser presents a user interface to the user that queries the user whether an upload is desired (block 320). Further, the user interface can include a field for indicating the data to be uploaded, or in some cases, the information to be uploaded is taken from a removable computer medium. Thus, in some cases where the applet is running on a programmer, the information on a floppy disk in the programmer is uploaded. In other cases, where the applet is running on a computer many files can be stored on a hard disk drive and uploaded simultaneously.

Once the data for upload is selected (block 320), the upload command is entered (e.g., a virtual button is clicked) (block 330). Where a single patient data file was selected for upload, the upload process is performed in a single pass. Alternatively, where multiple patient data files are selected for upload, a recursive process traversing a directory structure is completed until all of the patient information is uploaded. Various data is uploaded depending upon the type and model number of a particular implantable medical device, and the information provided by the patient and/or physician as previously discussed. Alternatively, information from the implantable medical device can be uploaded in one session, and other subjective and/or objective information uploaded in one or more other sessions.

In one particular embodiment where objective patient data, subjective patient data, and data from the implantable medical devices are all uploaded using a single upload request, the data can be automatically dispersed between system server 228 receiving data from implantable medical devices and a gateway server 242 receiving other subjective and/or objective data.

In one particular case, a thread is started to perform the actual upload to the server contacted via the Internet browser (block 350). In such a case, each upload of patient information begins with a “GET” request to the server indicating the start of directory upload (block 360). As an example, the serial number of the particular implantable medical device as well as the application model number and a date/time stamp can be passed as parameters to the “GET” request and used to create a file name. After the “GET” request is issued, a special “POST” request message can be sent to the server for each file being uploaded. The contents of the file are passed to the server in the body of the “POST” request, and the name of the file is passed as a parameter.

At least in some instances, privacy concerns are an issue and thus security in transferring information across the communication network is a concern. Security can be enhanced by configuring a servlet that distributes Java applets on the server side to run using a secure HTTP connection. This could be reflected on the user (e.g., client) side by the inclusion of a privacy indicator as is known in the art. Further, setting the HTTPS would take advantage of the previously suggested security. In addition to these approaches, authentication can be provided.

On the server side, two servlets handle the previously described “GET” and “POST” requests. In one particular case, the model number, serial number and/or date/time stamp can be used to define a unique path (e.g., directory location) for patient data associated with the device. If the path does not exist, then the servlet will attempt to create the path. As one example, the path may not exist where data about a particular patient is uploaded for the first time. During the original upload, the received information is archived for tractability and/or other future uses. Separate directories are used to save the uploaded files received at different times from the patient. These directories are children of the patient directory that was created earlier. The names of these directories can be assigned to reflect the running count of uploaded disks. As one example “1743_{—}200134/0” and “1743_{—}200134/1” and so on. These reflect the first and second uploads for the patient with a 1743 PG and a 200134 serial number. In one particular embodiment, the “/0” and “/1” are replaced with a received date/time stamp. Thus, where multiple transmissions from the same implantable medical device are received with the same date/time stamp, only one copy of the multiple transmissions is retained. The patient directory is saved as an attribute for the current session, and is made available for the servlet. The “POST” request causes the creation of a file in the current patient directory, and then copies the content of the “POST” request into the newly created file.

In some cases, if the upload is interrupted or otherwise fails, then an orphan directory with partial data is created on the server. This directory can be cleaned up by an administrative servlet. The administrative servlet could regularly scan an archive area of raw database 230 and delete any orphan directories. This servlet can use any log information maintained in the directory to determine the sessions that were interrupted.

When the upload is complete for a single patient directory, an indication of the completion is passed to the servlet. The servlet can then flag the directory as complete and also store any log information relevant to the upload to the directory. This log can capture any information about the upload including, but not limited to, the MAC (media access control) address or other indication of the machine that initiated the upload.

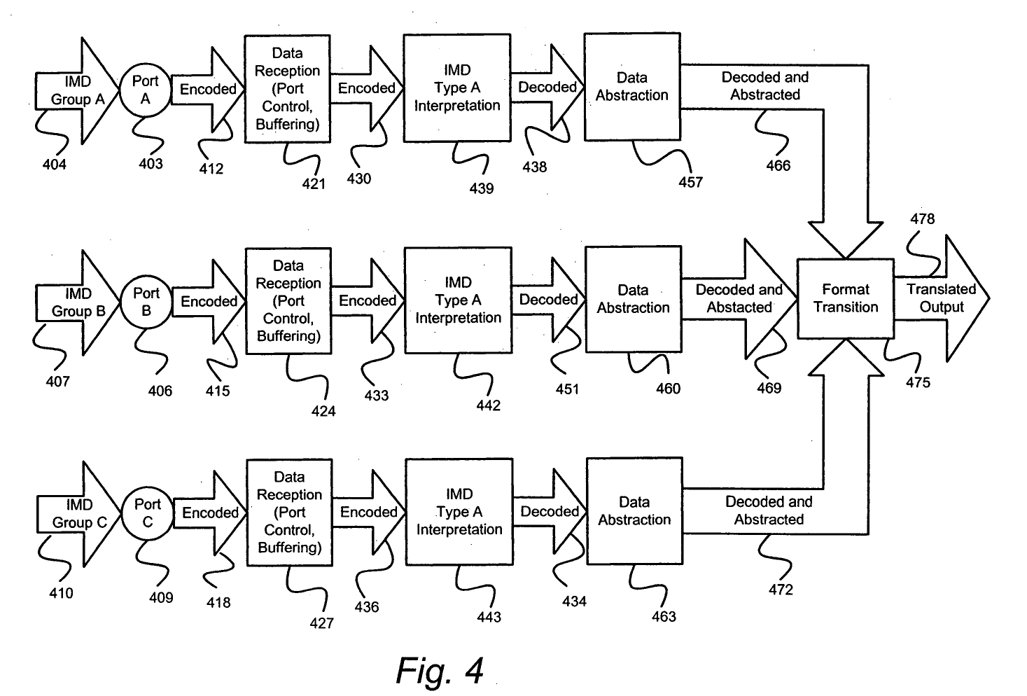

In some cases, the uploaded data is from an implantable medical device and as such can be in an encoded and/or encrypted form. In such cases, the data or a portion of the data is directed to a data translation system 232. Turning to FIG. 4 which depicts an embodiment of data translation system 232, three ports 403, 406, 409 are respectively dedicated to receiving data from particular groups of implantable medical devices 404, 407, 410. As a particular example, information from an implantable medical device included within implantable medical device group 404 is received by system server 228. In turn, system server 228 passes the received information from the implantable medical device to port 403. Where the information is from an implantable medical device from one of the other groups of implantable medical devices 407, 410 the received information is passed through another of ports 406, 409. It will be appreciated that the design of data translation system 232 is scalable and can be modified to provide translation, decoding, and/or decrypting of data from any number of implantable medical device groups. Thus, as will be appreciated, the number of implantable medical device groups illustrated is merely exemplary.

In one embodiment, a TCP/IP connection is opened to one of ports 403, 406, 409 with a request to convert data to be provided via the TCP/IP connection. As discussed, the port on which the data is received indicates the type of data that will be received (i.e., the type of implantable medical device from which the data originated). Thus, system server 228 is responsible for identifying the data type received, and for opening a TCP/IP connection with a port of data translation system 232 corresponding to the identified data type. Data translation system 232 can be implemented, for example, using “inetd”. The inetd daemon listens and accepts connections to multiple ports. When the inetd daemon accepts the connection, it spawns a conversion utility particular to the port (i.e., the implantable medical device) from which the data is received.

The received information 404, 407, 410 passes through the respective ports 403, 406, 409 as encoded data 412, 415, 418. As used herein, the term “encoded data” is used in its broadest sense and means data that is in a form specific to the implantable medical device from which it is derived. Thus, a particular pacemaker may produce encoded data of one format, while another pacemaker produces encoded data in another format. These two format types may be, for example, encoded data 412 and encoded data 415, respectively.

For illustration purposes, one type of encoded data derived from an exemplary implantable medical device is described herein. The provided encoded data is proprietary to the implantable medical device from which it is derived, and thus interpreting the encoded data includes determining the device type that produced the data. Based in part on the disclosure provided herein, one of ordinary skill in the art will recognize a number of different device types each producing a proprietary data set. Of course, one of ordinary skill in the art will recognize the following data type as exemplary, and not limiting. With this said, the exemplary encoded data includes ASCII header information as follows:

# TYPE:EPISODE SAVE DATE:02/25/04
# PROGRAMMER MODEL:XXXX SERIAL:0123456
# DEVICE MODEL:YYYY SERIAL:543210
# RAM VERSION:1.1 ROM VERSION:
# APPLICATION MODEL:ZZZZ VERSION:1.7
FORMAT_CODE,1
[ EPISODE EPISODE:1432 INDEX:87 ]
EpisodeNumber,“1,432”
EpisodeEgmShkChannel,1
EpisodeEgmVChannel,1
EpisodeStartDate,15-FEB-2004
EpisodeStartTime,16:11
EpisodeEndTime,01:47
EpisodeEndMessage,Data Overwritten
EpisodeInduced,Spontaneous
EpisodeInitialRate,142
EpisodeMeasuredOnsetPercent,0
EpisodeMeasuredOnsetTime,0
EpisodeProgrammedStabilityAcc,Off
EpisodeProgrammedStabilityInh,18
EpisodeProgrammedOnset,19
EpisodeProgrammedVFRate,190
EpisodeProgrammedVTRate,140
EpisodeProgrammedVT1Rate,--
EpisodeProgrammedSRD,1:00
EpisodeProgrammedOnsetLogic,And
EpisodeEgmAChannel,1
EpisodeProgrammedVGreaterARate,On
EpisodeProgrammedAFibThreshold,200
EpisodeATRTerminationMessage,
[ ATTEMPT EPISODE:1432 ATTEMPT:1 ]
AttemptNumber,1
AttemptPreAverageVRate,142
AttemptMeasuredStability,3
AttemptDetectionZone,VT Zone
AttemptTherapyAccelerateNote,
AttemptTherapyDelayNote,* Therapy Delayed until SRD
AttemptPostAverageVRate,143
AttemptTimestamp,01:03
AttemptPreAverageARate,141
AttemptAFib,False
AttemptVGreaterARate,False
AttemptPostAverageARate,140
AttemptATPBurstTimeOffset,01:23
AttemptATPName,VT ATP1 Ramp
AttemptATPTherapyDelivered,3 Bursts

The ASCII header information is followed by a number of encoded binary fields. As one example, the following encoded binary data (represented in ASCII character format) received from the implantable medical device describes the patient's heart functionality detected near the patient's atrium at or about the onset of an episode number 1432:

[image]

As another example of the encoded binary data received from the implantable medical device, the following data (again represented in ASCII character format) describes the patient's heart functionality detected near the patient's atrium following the episode number 1432.

[image]

In some cases, the encoded data derived from the implantable medical device includes an error detection and/or correction code (e.g., a checksum or CRC). This code can be used to determine if the data has been manipulated after being received from the implantable medical device, or if the data was not properly transmitted from the implantable medical device.

Encoded data 412, 415, 418 is buffered by a respective data reception block 421, 424, 427. Each of data reception blocks 421, 424, 427 releases one of buffered, encoded data 430, 433, 436, respectively. Buffered, encoded data 430, 433, 436 are respectively passed to a corresponding interpretation system 439, 442, 445. Each of interpretation systems 439, 442, 445 are designed to decode the respective buffered, encoded data 430, 433, 436. As used herein, the term “decode” is used in its broadest sense and implies any process whereby the encoded data is translated or otherwise modified to conform to a desired format.

As one example of decoding, buffered, encoded data 430 may include a portion of an implantable medical device identification number spread across a number of bits within a data stream. In such a case, interpretation system 439 can be responsible for reassembling the dispersed bits into, for example, two byte words. Alternatively, or in addition, the previously described illustrative data may be parsed and decoded to yield a variety of data fields. For example, the device type, model number, serial number, patient name, lead impedance, episode time and date stamps, and a large number of other device parameters can be decoded from the encoded binary data.

The decoded data 448, 451, 454 is passed to respective data abstraction engines 457, 460, 463. Data abstraction engines 457, 460, 463 each associate particular elements of the respective decoded data 448, 451, 454 with global fields common to data received from implantable medical device groups 404, 407, 410. Data abstraction engines 457, 460, 463 respectively provide decoded and abstracted data sets 466, 469, 472. In some cases, decoded and abstracted data sets 466, 469, 472 are passed to a format translation engine 475 which provides a translated data output 478.

In one particular embodiment, data abstraction engines 457, 460, 463 assemble decoded data 448, 451, 454 into an XML flat file format, and format translation 475 is a pass through. In other embodiments, the XML format can be translated into a particular spread sheet format, or other suitable format. One such example of an XML format decoded and abstracted data set derived from the previously discussed encoded binary data is as follows:

<parameter name=“PatientName”>JONES, SMITH</parameter>
<parameter name=“PrmRtcDateTime”>25-FEB-2004 18:35</parameter>
<parameter name=“SystemModelNumber”>H135</parameter>
<parameter name=“ProgrammerModel”>XXXX</parameter>
<parameter name=“ProgrammerSerialNumber”>0123456</parameter>
<parameter name=“SystemSerialNumber”>YYYY</parameter>
<parameter name=“SystemFirmwareRevision”>1.1</parameter>
<parameter name=“ProgrammerApplicationModelNum”>
ZZZZ</parameter>
<parameter name=“ProgrammerApplicationRevision”>1.7</parameter>
<parameter name=“EpisodeNumber”>1,432</parameter>
<parameter name=“EpisodeStartDate”>15-FEB-2004</parameter>
<parameter name=“EpisodeStartTime”>16:11</parameter>
<parameter name=“EpisodeInduced”>Spontaneous</parameter>
<collection name=“C_EGRAM_ONSET”>
Onset EGM (10 sec max)
<traces frequency=“400 hz”>
<egram_data source=“atrium” samples=“4000”>8202, 8202, 8197, 8192,
8180, 8169, 8174, 8179, 8185, 8192, 8198, 8205, 8207, 8209, 8214, 8219,
8299, 8380, 8076, 7772, 7973, 8175, 8231, 8287, 8292, 8298, 8270, 8243,
8224, 8205, 8193, 8182, 8175, 8169, 8167 {. . . .}
<collection name=“C_EGRAM_POST_ATTEMPT”>
Post-attempt EGM (10 sec max)
<traces frequency=“400 hz”>
<egram_data source=“atrium” samples=“2774”>8192, 8192, 8192, 8192,
8192, 8192, 8190, 8189, 8190, 8192, 8190, 8189, 8190, 8192, 8192, 8192,
8190, 8189, 8192, 8195, 8193, 8192, 8192, 8192, 8190, 8189, 8199, 8209,
8280, 8352, 8089, 7826, 8002, 8179, 8229 {. . . .}

In an embodiment of the invention, each of interpretation engines 439, 442, 445 are implemented in software. Further, in some cases, at least significant portions of the software is the same as that implemented in a programmer specific to a particular implantable medical device or group of implantable medical devices. Thus, development effort required to create data translation system 232 is greatly reduced. Further, the scalability of such a system is increased as software from a device specific programmer can be ported and/or recompiled for use in data translation system 232. In addition, a pipe or thread (e.g., a combination of port 403, data reception block 421, and data abstraction engine 457) can be assembled around an interpretation system (e.g., interpretation system 457) comprised of the ported and/or compiled software.

In some cases, the decoded and abstracted data is passed back to system server 228, where it is stored to comprehensive database 238 in XML format or in a particular database format, as discussed below. The converted files can be given unique filenames, since they correspond to bank and episode files, and therefore, all the files can be saved directly under patient directory or record, and there is no need for another level of directories. For example, “1743_{—}200134/00001234.eps” and “1743_{—}200134/00abde87.bnk”. In various cases, translated output data 478 is passed back to system server 228 where it is stored to comprehensive database 238, while in other cases, the translated output data is passed back to system server 228 where it is transferred directly to a requestor or a requestor system.

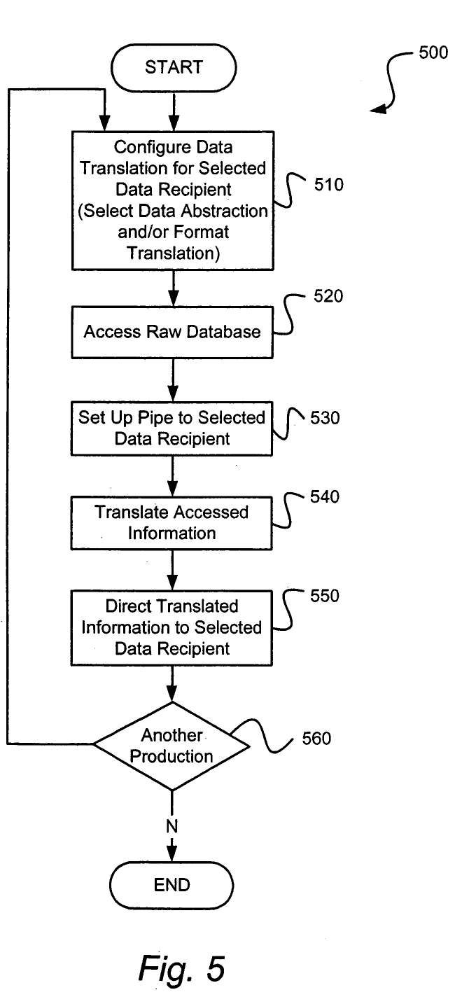

Turning to FIG. 5, a flow diagram 500 illustrates one method in accordance with some embodiments of the present invention for real time processing of data from raw database 230 based on particular requirements. Following flow diagram 500, data translation system 232 is configured to prepare data for a selected recipient (block 510). This includes indicating a number of data fields known to data abstraction engines 457, 460, 463, and a particular desired format known to format translation engine 475.

Data is then pulled from raw database 230 and passed to a particular one of ports 403, 406, 409 based on the type of implantable medical device from which the data was taken (block 520). As previously discussed, an application associated with the particular type of raw data is spawned setting up the pipe through which the data will be processed (e.g., data reception block 421, interpretation system 439, data abstraction engine 457, and format translation system 475) (block 530). The information is then translated as previously discussed from raw, or encoded data to the translated data output (block 540). The translated data is then directed to the desired recipient and/or repository (block 550).

As previously discussed, in some cases all of the raw information is translated (i.e., decoded) and directed back to comprehensive database 238. In other cases, only a portion of the raw information is translated and redirected to a recipient and/or repository such as, for example, subset databases 248, 252. In such cases, a command is provided indicating the information that is to be included with the output (to be used by data abstraction engines 457, 460, 463) and the format in which the output is to be delivered (to be used by format translation engine 475).

Data abstraction engines 457, 460, 463 receive the decoded information 448, 451, 454 but only the portions of the information designated by the command are retained by data abstraction engines 457, 460, 463. This portion of the information retained is passed to format translation engine 475, where the data is formatted in a selected format. In some cases, where the desired format is the same as that provided by data abstraction engines 457, 460, 463, format translation engine 475 merely passes the data through. Thus, for example, where data translation engines provide the data in an XML format and the desired format is XML, no format translation is performed. Alternatively, where another format such as a particular spreadsheet format is desired, the data from data abstraction engines 457, 460, 463 is formatted into the spreadsheet format. The information is then passed back to system server 228, which in turn passes it to the requesting entity and/or a designated subset database.

With the translation complete, it is determined if another data production is to be performed (block 560). If an additional production is to be done (block 560), the raw data is accessed, translated, and provided to another entity consistent with a received command (block 510) as previously described. Alternatively, where no additional production is to be done (block 560), the process ends.

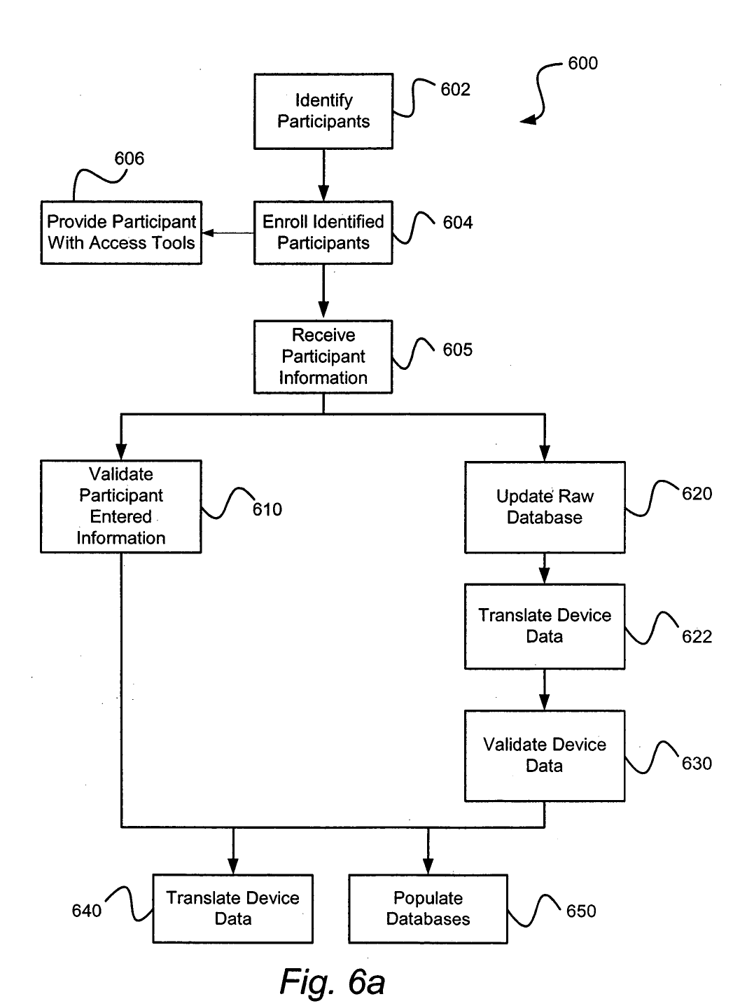

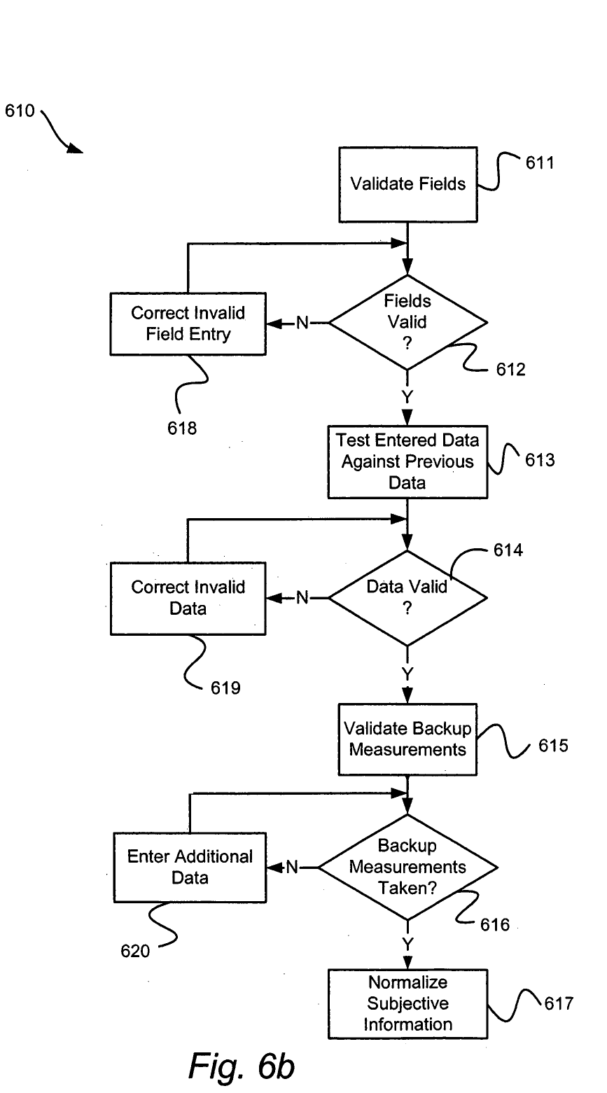

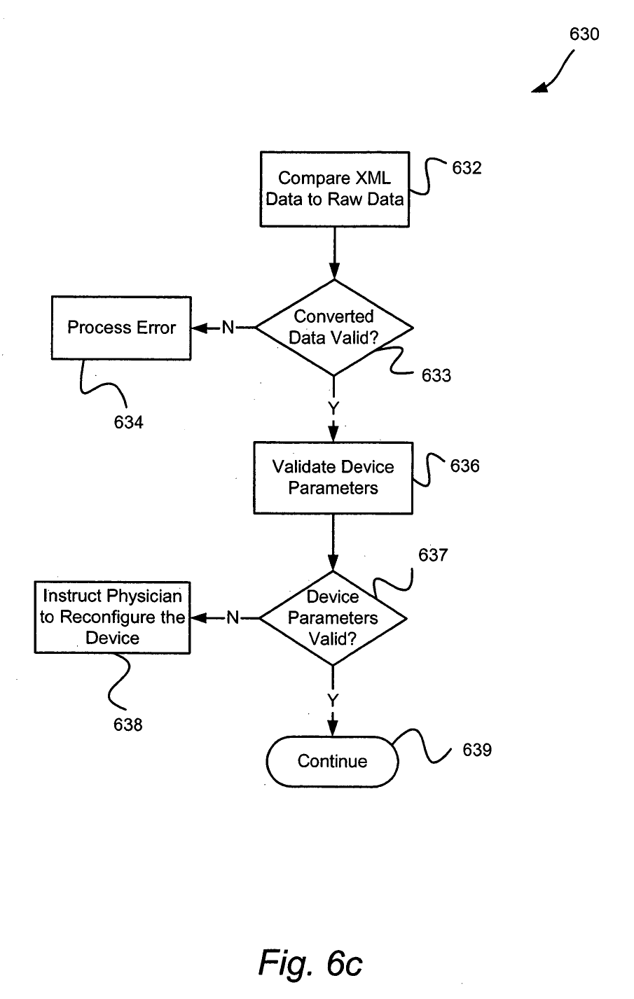

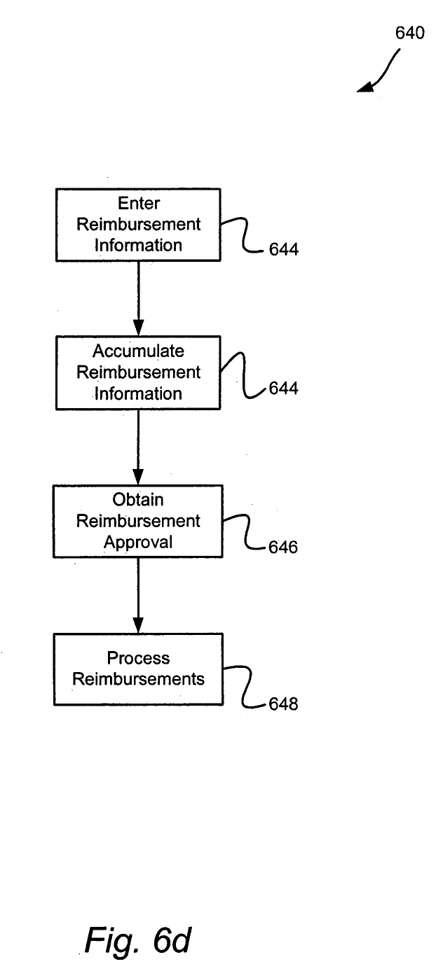

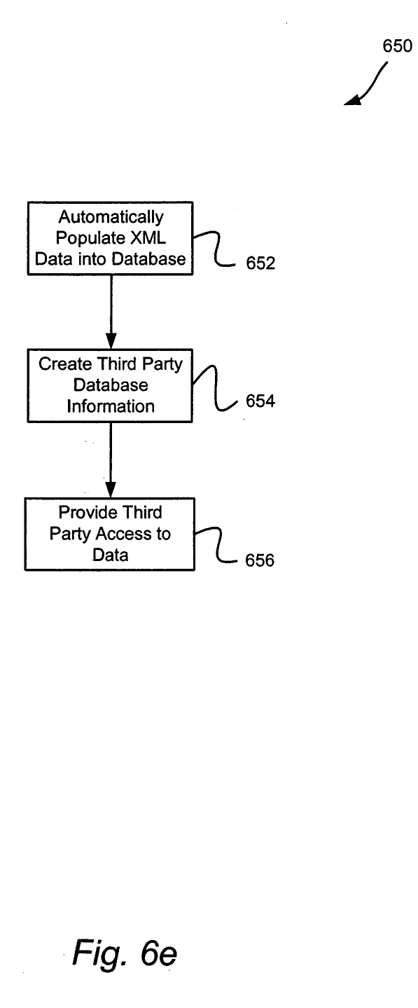

Turning to FIG. 6a, a flow diagram 600 illustrates a method for performing clinical and operational trials using system 100 in accordance with some embodiments of the present invention. Following flow diagram 600, various participants are identified (block 602). In a typical scenario, one or more physicians that typically implant a type of class of implantable medical devices are chosen. However, based on the disclosure provided herein, one of ordinary skill in the art will recognize a number of other “participants” including, for example, recipients of a given medical device.

These participants are enrolled in the study (block 604). This enrollment includes downloading or sending one or more software programs or other access tools that allow the participant to access the system (block 606). Further, enrollment includes receiving a variety of information about the participant that can be received via a communication network or via regular mail. In particular cases, this information is provided using one or more of the software programs provided when the participant enrolls. This enrollment information can include the participant's name and contact information. Further, in some cases, the participants are reimbursed for their participation, thus enrollment can include obtaining taxreimburseer information, direct deposit information, and the like. Based on the disclosure provided herein, one of ordinary skill in the art will recognize a number of other enrollment data that may be gathered about a participant.

Once the participant is enrolled, various information can be gathered via the participant or a participant system (block 608). This information can be, for example, information received from an implantable medical device read using a device programmer or other device monitor, as discussed above. In addition, the participant can enter various information, such as subjective data, objective data, other patient information, or the like. The received information can be processed separately depending upon the type of the information. For example, the participant-entered information can be validated to make sure the information was entered correctly (block 610). If the information is not entered correctly (block 611), the system will notify the participant to fix errors (block 612) This validation process is more fully described below with reference to FIG. 6b.

In addition, the received medical device information is stored to raw database 230 as described above (block 620). Because the data received from the implantable medical devices is encoded, the encoded data is passed to data translation system 232, where it is translated as described above in relation to FIG. 4 (block 622). The resulting translated information then is validated (block 630) to ensure that the translation occurred properly and to ensure that the implantable medical devices are configured properly. This validation process is discussed in more detail below with reference to FIG. 6c.

After both the participant-entered information and the uploaded medical device information is validated, the system processes a reimbursement to the participant (block 640), and stores the validated data in comprehensive database 238 (block 650). In addition, various portions of the collected information can be dispersed to other associated databases.

Referring now to FIG. 6b, one embodiment of a method for validating participant-entered information (block 610) will be discussed in more detail. As discussed above, during or just after a patient visit, a physician (or an assistant or data entry service) enters information about the patient, including objective information, subjective information, or any other medical or diagnostic information that may be relevant about the patient. In one embodiment of the invention, the physician is provided with a data entry screen, which prompts the physician to enter at least some of the patient information. The data entry screen on the physician's computer system, or it can run on a mobile data entry device, such as a PDA, cellular phone, or some other mobile device. Also, the data entry screen can be any suitable data entry program, such as a device resident program, or an Internet web page or applet downloaded to the physician's device.

After the physician enters data into the data entry screen or program, the data fields are validated to ensure that data entry is correct (block 611). The data entry validation can include many different types of validation techniques. For example, for objective data entry, the data entry validation can make sure height, weight, or other entered objective measurements are reasonable. One example might be to compare the height, weight or other variables against reasonable ranges. Also, diagnosis information can be validated to ensure proper diagnosis information is entered. For example, an entered medical term might be checked to ensure that the term actually exists, or an entered diagnosis might be checked to ensure that it is reasonable based on other entered data. As one skilled in the art will appreciate, there could be a number of different ways to validate the data entry fields, and thus the present invention is not limited to any particular validation process or technique.

Also, the field validation process can be performed by the data entry device at the physician's location, or it can be performed by a centralized processing system, such as the data collection system 206 or the central data processing and repository system 208 in FIG. 2. In the latter example, the entered data is transmitted from the physician's system to the centralized processing system, for example, via a web page or applet and validated there. In both cases, however, the validation process is real-time, and the physician or data entry person is notified of errors relatively quickly (block 612).

If data entry errors occur, the physician or data entry person is prompted to correct the error and resubmit the information (block 618). Otherwise, if the data appears to be entered correctly, it is transmitted to a centralized processing system, such as the data collection system 206 or the central data processing and repository system 208 in FIG. 2. At the centralized processing system, the participant-entered data enters a second validation process, which includes testing the entered data against previously entered or recorded data (block 613). In accordance with this aspect of the invention, the entered data is compared to data in comprehensive database 238 or subset database 248 as a back-up validation.

Again, any number of different validation checks can be performed. For example, entered height, weight or other patient demographic information might be checked against previously entered data to see if it is reasonable. If the patient's height or weight changed significantly from previous visits, the physician might be prompted to verify the entered information. Also, if the newly entered diagnosis information is inconsistent with a previous diagnosis, the system might inform the physician. If the system detects validation errors, it notifies the physician or data entry person of the error (block 614). Then the physician can either fix the error or confirm that the entered data is correct (block 618).

In order to obtain the most accurate information possible, it sometimes is beneficial to perform multiple or back-up measurements. Thus, in one embodiment, the system can be configured to prompt the physician to perform additional or back-up measurements for at least some of the fields (block 615). If the system requests back-up measurements, but the physician has not entered them, the system will prompt the physician to enter the additional measurement data (block 619). They physician can elect to enter the additional measurements (block 619), or the physician can elect not to enter the additional measurement. If the additional measurements are not entered, the system can flag the measurement data as being less reliable, or the system can add a weighting factor to the measurement data that indicates that the measurement data may have errors or is less reliable than data with back-up measurement.

Finally, the subjective information entered by a physician can be normalized to remove or compensate for any physician biases that might occur (block 617). As one skilled in the art will appreciate, subjective information is just that, subjective, and physicians will have different perspectives on various medical, emotional and other factors based on their own beliefs or emotional states. For example, some doctors consistently might have negative views of certain subjective analyses, while other doctors consistently might have positive views of the same analyses. Thus, in accordance with one embodiment of the present invention, the system might adjust or normalize certain subjective data based on known physician biases. Alternatively, the system might determine a statistical average for subjective information entered by a number of physicians, and discard any information that is not within the a range of the average. There are a number of different ways entered data can be normalized based on a number different factors, and thus, the present invention is not limited to the examples set forth herein.

As discussed above, the implantable medical device data is transmitted to central data processing and repository system 208 and stored in raw database 230 (block 620). The implantable medical device information then is decoded into, for example, XML format as described above in relation to FIG. 4 (block 622). The resulting translated or decoded XML data then is validated (block 630) to ensure that the translation occurred properly and to ensure that the implantable medical device is configured properly. Referring now to FIG. 6c, one embodiment of a method for validating the translated medical device data will be discussed in more detail. In the illustrated embodiment, the translated XML data is compared to the raw data stream (block 632). In accordance with this aspect of the invention, a majority of the data in the raw data stream is in ASCII format. Thus, to validate the translation, the system compares the XML data with the ASCII data to determine if the data was translated properly (block 633). If errors are found, the system can either fix the data fields with errors, or re-run the translation process to fix the errors (block 634). Also, if errors are found, it may be necessary to troubleshoot and fix the translation process before proceeding.

After it is determined that the translated XML data is error free, the system validates the implantable medical device configuration (block 636). As mentioned above, one application of the systems and methods of the present invention is to monitor and manage clinical trials, for example, in order to obtain FDA approval for a device or to test various applications of the device. Thus, in order to have a successful clinical trial, it is important that the physicians configure the implantable medical devices in accordance with the clinical trial parameters. Also, in some instances, even if a device is not being analyzed as part of a clinical trial, it still might be possible to validate the device configuration, for example, by checking to ensure that the device configuration is proper for a patient's diagnosis or medical condition.

In accordance with this aspect of the invention, the system analyzes various medical device parameters from the medical device data stream to ensure that the physician configured the medical device properly (block 637). For example, the system can check the medical device parameters against a predefined clinical trial configuration to verify that the device is configured correctly. If the device is not configured correctly, the system can instruct the physician to make the appropriate changes, so that the device conforms to the clinical study parameters (block 638). As discussed in more detail below, the system can delay clinical trial reimbursements to physicians until the device(s) is configured correctly. Thus, one significant benefit of the present invention is that it can provide immediate medical device validation feedback to a physician, so that the physician can make appropriate changes to the implantable medical device while the patient is still in the physician's office, thus facilitating a better, more accurate clinical study and quicker reimbursement approval for the physician's services. After the implantable medical device data has been validated, the system will continue with the database population and participant reimbursement procedures (block 639).

In accordance with an alternative embodiment, the system can be configured to intelligently provide diagnosis advice. For example, the system might analyze a patient's medical condition and history and determine that an implantable medical device is not optimally configured for the patient and his/her condition. If this occurs, the system can provide device configuration and other diagnosis advice, which the physician may or may not follow.

In prior art systems, it can be cumbersome and difficult to process reimbursements to physicians for participating in clinical trials. Every time a physician sees a patient, the physician must submit paperwork requesting reimbursement. Before the physician is reimbursed, the compensating entity (generally, a medical device manufacturer) must validate the physician's work to verify that he/she is complying with the clinical study requirements. If clinical study requirements are not being met, the compensating entity must inform the physician of changes that need to be made. A significant problem with the old system is that it can be weeks, if not months, before a physician is notified of a problem. Because of the delay, it is difficult to obtain the necessary information to fix the problem. Indeed, many times a physician must re-visit the patient to fix medical device configuration errors or obtain new patient information. This is very time consuming and expensive. Also, after the follow-up visit, the physician again must submit paper work for reimbursement, and the process starts over.

If, on the other hand, everything is proper, the compensating entity manually prepares a check request, which must be approved before reimbursement is issued. The check request typically requires an individual to manually complete paperwork by entering reimbursement information, such as name, address, phone numbers, tax ID, department being charged, amount, and the person to approve the request. The request then goes to the approval authority who signs-off on the request. If approved, the request then goes to accounting for processing, which generally requires the data to be manually entered into a system so that a check can be cut. The compensating entity must follow this procedure for every patient of every physician in a clinical study, which can number in the thousands for each study. As a result, it can cost medical device manufacturers tens, if not hundreds, of thousands of dollars each year to process these check requests.

The systems and methods of the present invention automate this process. First, as discussed above, the medical information (including the medical device information) automatically is uploaded to central data processing and repository system 208 and validated in real time or near real time. As a result, if there are data errors or procedural errors, the system can instruct the physicians on how to rectify the problems. In some instances, the errors can be rectified while a patient still is at the physician's office, thus reducing the need for follow-up visits to fix mistakes. Also, after the data has been validated, the system of the present invention is adapted to automatically process participant reimbursements, thus eliminating the need for the manual procedures.

Referring now to FIG. 6d, one embodiment of a method for processing participant reimbursements (block 640) will be discussed in more detail. First, after a physician completes a reimbursable task (e.g., performing a device implant procedure; conducting follow-up exams, entering clinical trial data into the system, etc.), and the task is validated, the system automatically enters reimbursement information into a database (block 642). In some embodiments, the reimbursement information can include physician reimbursement information, the procedure performed, the reimbursement amount, or the like. Because much of this information probably already is stored in the system database, it can be pulled from the database and populated into an automatic reimbursement record for processing. For example, since the physician ID is known, the physician's address, phone numbers, tax ID, clinical trial information, etc. can be pulled from comprehensive database or some other database and used in the reimbursement record.

The system can be configured to process the reimbursement record immediately, or the reimbursement records can be accumulated and processed on a periodic basis, for example monthly or quarterly (block 644). Regardless of the reimbursement frequency, reimbursement records for multiple physicians can be accumulated and forwarded for approval (block 646). In this manner, the approval authority can review and approve hundreds of reimbursement requests at one time, instead of each one individually, as is done in the prior art systems. In accordance with this aspect of the invention, the reimbursement records are accumulated into an electronic report or form and forwarded to the approval authority electronically. In one embodiment, the reimbursement records can be formatted into an EXCEL® spreadsheet and forwarded via an email. In other embodiments, other electronic reports or communication means can be used. One skilled in the art will appreciate that the present invention is not limited to any particular reporting format or communication process.

After reimbursements are approved, the reimbursement information is submitted electronically to an accounting system for processing (block 648). Again, any communication process can be used. In one embodiment, the reimbursement information is converted to an EXCEL® spreadsheet and forwarded to the accounting system, which, in turn, loads the data from the spreadsheet into the system for processing. Alternatively, the reimbursement information can be converted into HTML, XML, or some other format and forwarded to the accounting system. In still another embodiment, the accounting system might be compatible with the system database, and thus, it can obtain the information directly from the database using a SQL call, or the like. Regardless of how the accounting system receives the data, upon receipt it processes the reimbursements and prepares checks accordingly. In addition, the system can be configured to prepare reimbursement reports for review, and it may be configured so that the physicians can access the system to determine what they are due. Thus, in at least some embodiments of the present inventions, a push-button approach allows for extraction of validated reimbursement data for each physician/practice versus previous manual preparation of each reimbursement; allow for reimbursement on a daily to quarterly (potentially 6 months to annual too) basis per physician/practice; allow for approval of multiple reimbursements versus on a per reimbursement basis; and where processing is done in batch mode, potential duplicate reimbursements can be flagged and reconciled. In some embodiments, the reimbursement information can be extracted from a database and flag set in association with each record indicating a reimbursement processed in relation to the record. For future reimbursements, the flag is checked to determine if a reimbursement has already been processed.

As discussed above, after the data (i.e., objective information, subjective information and implantable medical device information) is validated it is automatically populated into one or more databases, such as comprehensive database 238 and/or subset databases 248, 252. The process of populating the one or more databases is illustrated in FIG. 6e. In accordance with the illustrated embodiment, after a particular patient's objective data and the subjective data is validated, it is populated into a database record associated with the patient name or other identification. The manner in which the data is populated into the database is dependent upon the particular database being used and upon the format in which it receives the data. In one embodiment, the data is received in XML format and populated into the database record in a know fashion; i.e., a program extracts data from the XML tags and populates it into corresponding database record fields. As one skilled in the art will appreciate, any database system can be used, such as, for example, SAP, Oracle, People Soft, JD Edwards, Microsoft SQL Server, SAS, or the like. In one embodiment, the SAS database is used.

In addition, after the implantable medical device data is validated, it too is populated into comprehensive database 238. In accordance with this aspect of the invention, the implantable medical device data is transferred from its XML format to a database record. Before it is loaded into the database record, however, it is marked with patient identifier and a date and time stamp. The patient identifier can be any suitable identifier, such as name, patient ID number, medical device ID number, or the like. Also, the date and time stamp is used so that multiple device downloads from a single day can be loaded into the system. The time stamp distinguishes the downloads. Similarly, the date stamp is used to distinguish between down loads from different days. In this manner, the database can maintain separate and distinct records for each medical device download.

Further, in accordance with another aspect of the present invention, it is possible that implantable medical device information from a first download is the same as the device information from a subsequent download, for example, when a pulse generation device, such as a defibrillator, or the like, generates no new pulses. Thus, when this occurs, the subsequent device download data is not saved, because it would be redundant information.

After data is populated into comprehensive database 238, some or all of that data can be made available to third parties, as discussed above. In one embodiment, the system can create one or more third party databases, such as subset databases 248, 252, with some of all of the available data (block 654). The subset databases could be maintained on central data processing and repository system 208, or they could be maintained at other locations, such as data collection system 206 and/or diagnostic system 210. For example, the data could be transmitted to those systems and populated into the subset databases by the systems. In addition, after the subset databases are created, security and control features can be implemented to control access to the data in the database (block 656). Thus, in this manner, HIPPA control requirements can be met.

Further, in accordance with other embodiments of the invention, instead of creating new databases for access by third parties, comprehensive database 238 can be configured with security and access control features that allow the third parties access to approved data, but yet prohibit access to other unauthorized data. Thus, this could be another method for controlling data access, and thus implementing HIPPA requirements.

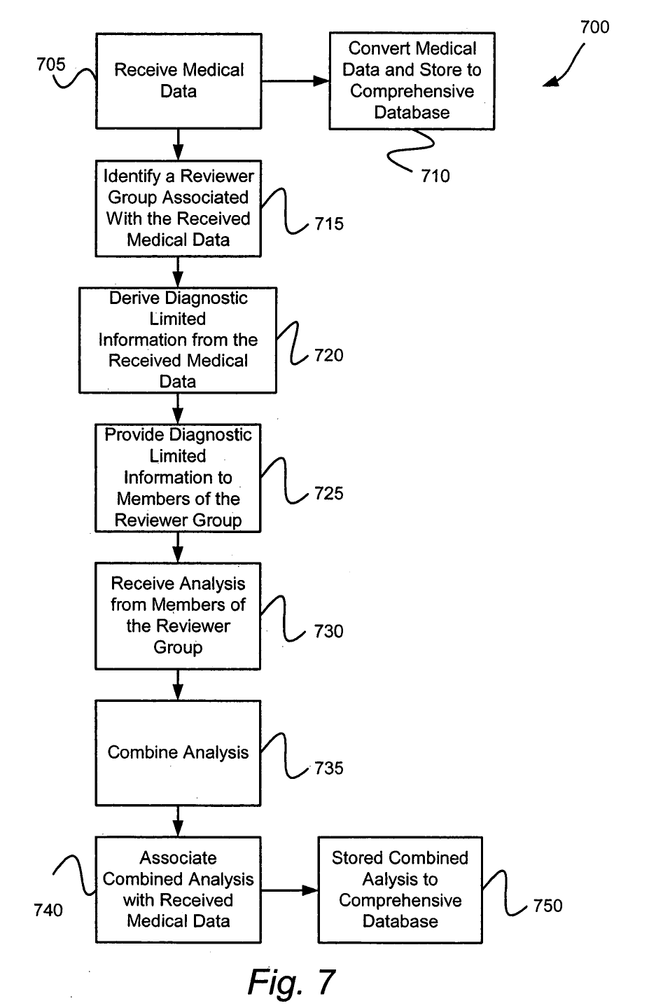

Turning to FIG. 7, a flow diagram 700 illustrates a method for obtaining medical analysis in accordance with some embodiments of the present invention. Following flow diagram 700, a medical data set is received (block 705). Such a medical data set can be received from a physician or other participant enrolled in a study as previously discussed in relation to flow diagram 600. This medical data set is processed as previously discussed in relation to blocks 630-690 and is ultimately placed in comprehensive database 238 (block 710). As will be appreciated, the data could be placed in an alternative or an additional database, such as subset database 252 associated with diagnostic system 210.

In addition, a reviewer group associated with the received medical data is identified (block 715). The reviewer group is a collection of one or more individuals or entities capable of receiving a portion of the medical data set, and returning an analysis of the portion of the medical data set. As one example, the reviewer group may include one or more physicians, specialists, and other trained medical personnel capable of providing a medical diagnosis based on the portion of the medical data set. Based on the disclosure provided herein, one of ordinary skill in the art will recognize a variety of possible participants that can be included in the reviewer group.

A diagnostic limited data set is derived from the received data set (block 720). This process prepares the data for transmission to a reviewer and can include the incorporation of data originated from an implantable medical device, physician provided subjective information, physician provided objective information, and/or the like. Where a reviewer is interested in only a subset of the received medical data set, the subset of interest is distilled and maintained as a diagnostic limited data set. Further, in some cases, it may be desirable to exclude information capable of identifying the patient to which the received medical data set is associated from the diagnostic limited data set. This diagnostic limited data set is then communicated to each of the reviewers via communication network 202 (block 725).

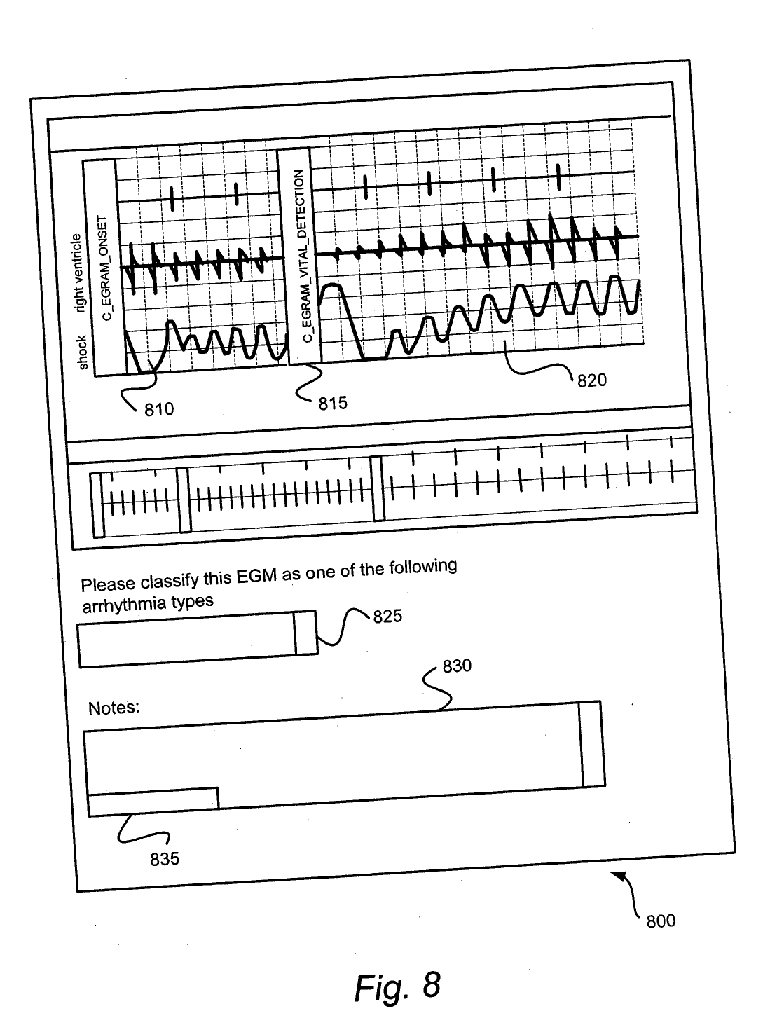

FIG. 8 provides an example of a diagnostic limited data set presented in graphical format. Turning to FIG. 8, a user interface 800 is displayed on a receiver, such as data input devices 222, 258 associated with the reviewers. As illustrated, user interface 800 includes a diagnostic limited data set presented as an electrocardiogram. This particular electrocardiogram can be derived from the EGRAM_ONSET data 810, episode occurrence point 815, and POST_EGRAM data 820 that was previously described as exemplary data in relation to FIG. 4 above. Further, user interface 800 includes an input field 825 where the reviewer can insert a diagnosis based on the electrocardiogram data, and an input field 830 where the reviewer can provide additional notes and analysis.

Returning to FIG. 7, the reviewer analyzes the provided diagnostic limited data and provides an analysis thereof (block 730). This analysis can be, for example, in the form of a medical diagnosis and other notes as described in relation to FIG. 8. The analysis is received and combined with corresponding analysis from other members of the group of reviewers (block 735). Thus, where there are ten reviewers and all ten reviewers report the same medical diagnosis, the analysis can be reduced to a single indication of the medical diagnosis. In such a case, a note may be added indicating that all ten reviewers agreed. Where notes or analysis is provided by a reviewer that is different from other reviewers, it is maintained. Where all of the reviewers disagree, a combination may not be possible and thus is not performed. The analysis data is associated with the medical data set to which it corresponds (block 740), and stored relative to the corresponding medical data set (block 750).

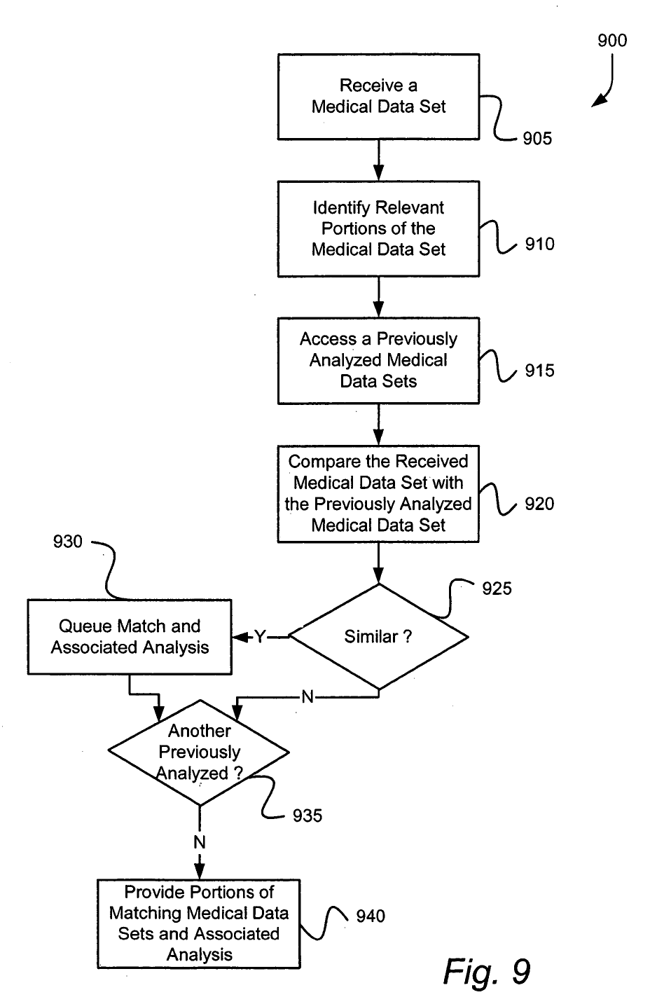

Turning now to FIG. 9, a flow diagram 900 illustrates a method for providing a diagnosis or other analysis based on a medical data set in accordance with some embodiments of the present invention. Following flow diagram 900, a medical data set is received (block 905) along with a request for a diagnosis. This medical data set can be received, for example, from a patient's physician who is seeking additional guidance on the patient's condition. Relevant portions of the received medical data set are then identified (block 910). This can include removing portions of the medical data set that are unrelated to a diagnosis being requested. As one particular example where a diagnosis related to human heart functionality is requested, the relevant portion of the medical data set may include an electrocardiogram.

One or more previously analyzed medical data sets are accessed from, for example, comprehensive database 238 or subset database 252 (block 915), and portions of the previously analyzed medical data sets corresponding to the identified relevant portions of the received medical data set are compared (block 920). Thus, for example, a received electrocardiogram may be compared to electrocardiogram associated with the previously analyzed medical data set. It is determined if the compared data portions are similar (block 925), and where they are similar the portion of the previously analyzed data set compared is queued in a temporary memory area along with analysis information maintained in relation to the previously analyzed medical data set (block 930).

It is determined if additional previously analyzed medical data sets are to be considered (block 935). Thus, for example, the process may continue until a single positive comparison is found, until a preset number of positive comparisons are found, or until all previously analyzed data sets have been considered. Where additional previously analyzed data sets are to be considered (block 935), blocks 915-930 are repeated. Alternatively, the process completes by communicating relevant portions of the matched previously analyzed data sets along with corresponding analysis information to the requestor (block 940). As previously discussed in relation to FIG. 7, the analysis information can be, for example, medical diagnosis information.

In conclusion, one or more embodiments of the invention now have been described in detail for purposes of clarity and understanding. However, it will be appreciated that certain changes and modifications can be practiced within the scope of the appended claims. Thus, although the invention is described with reference to specific embodiments and figures thereof, the embodiments and figures are merely illustrative, and not limiting of the invention. Rather, the scope of the invention is to be determined solely by the appended claims.

1. A system for translating medical data, the system comprising:
a first interpretation system, wherein the first interpretation system is operable to receive a first encoded data set received from a first implantable medical device and to provide a first decoded data set;
a second interpretation system, wherein the second interpretation system is operable to receive a second encoded data set from a second implantable medical device and to provide a second decoded data set;
a first data abstraction engine, wherein the first data abstraction engine is operable to receive the first decoded data set from the first interpretation system;
a second data abstraction engine, wherein the second data abstraction engine is operable to receive the second decoded data set from the second interpretation system; and
wherein the first data abstraction engine and the second data abstraction engine provide a first abstracted data set and a second abstracted data set, respectively, in a common data format.

2. The system of claim 1, wherein the system further comprises:
a first communication link, wherein the encoded data set received from the first implantable medical device is received via the first communication link; and
a second communication link, wherein the encoded data set received from the second implantable medical device is received via the second communication link.

3. The system of claim 2, wherein the first communication link is a server port.

4. The system of claim 2, wherein the system further comprises a system server, wherein the system server includes a processor and a computer readable medium, and wherein the computer readable medium includes instructions executable by the processor to:
receive the first encoded data set from the one of a plurality of implantable medical device types via a communication network;
identify the one of the plurality of medical device types; and
communicate the first encoded data set via the first communication link to the first interpretation system.

5. The system of claim 4, wherein the computer readable medium further includes instructions executable by the second processor to:
store the first encoded data set to a raw database.

6. The system of claim 4, wherein the computer readable medium further includes instructions executable by the processor to:
receive the first abstracted data set;
receive the second abstracted data set; and
store the first abstracted data set and the second abstracted data set in a comprehensive database.

7. The system of claim 4, wherein the computer readable medium further includes instructions executable by the processor to:
receive the first abstracted data set;
receive the second abstracted data set;
distribute at least a portion of the first abstracted data set and the second abstracted data set to a first recipient; and
distribute at least a portion of the first abstracted data set and the second abstracted data set to a second recipient.

8. The system of claim 7, wherein the first recipient is a first subset database, and the second recipient is a second subset database.

9. The system of claim 7, wherein the first recipient is selected from a group consisting of:
a gateway server; and
a diagnostic server.

10. The system of claim 1, wherein the common data format is a standardized format.

11. A system for translating medical data, the system comprising:
a data translation system, wherein the data translation system comprises a processor and a computer readable medium, and wherein the computer readable medium includes instructions executable by the processor to:
receive an encoded data set from one of a plurality of implantable medical device types via one of a plurality of ports, wherein each of the plurality of ports is assigned to one of the implantable medical device types;
select a conversion utility, wherein selection of the conversion utility is based at least in part upon the port via which the encoded data set is received from the one of the implantable medical devices;
spawn the conversion utility; and
translate the encoded data set to a decoded data set.

12. The system of claim 11, wherein the processor is a first processor, and wherein the computer readable medium is a first computer readable medium, wherein the system further comprises a system server, wherein the system server includes a second processor and a second computer readable medium, and wherein the second computer readable medium includes instructions executable by the processor to:
receive the encoded data set from the one of a plurality of implantable medical device types via a communication network;
identify the one of the plurality of medical device types; and
direct the encoded data set to the one of the plurality of ports corresponding to the one of the plurality of implantable medical device types.

13. The system of claim 12, wherein the second computer readable medium further includes instructions executable by the second processor to:
store the encoded data set from the one of the plurality of implantable medical device types to a raw database.

14. The system of claim 11, wherein the computer readable medium further includes instructions executable by the processor to:
abstract the decoded data set to an abstracted data set with elements common to each of the plurality of implantable medical device types.

15. The system of claim 14, wherein the computer readable medium further includes instructions executable by the processor to:
communicate the abstracted data set to a recipient selected from a group consisting of: a system server, a gateway server, and a diagnostic server.

16. The system of claim 15, wherein the processor is a first processor, and wherein the computer readable medium is a first computer readable medium, wherein the system server includes a second processor and a second computer readable medium, and wherein the second computer readable medium includes instructions executable by the processor to:
receive the abstracted data set; and
store the abstracted format data set to a comprehensive database.

17. The system of claim 15, wherein the processor is a first processor, and wherein the computer readable medium is a first computer readable medium, wherein the system server includes a second processor and a second computer readable medium, and wherein the second computer readable medium includes instructions executable by the processor to:
receive the abstracted data set; and
distribute at least a portion of the abstracted data set to a recipient.

18. The system of claim 15, wherein the processor is a first processor, and wherein the computer readable medium is a first computer readable medium, wherein the system server includes a second processor and a second computer readable medium, and wherein the second computer readable medium includes instructions executable by the processor to:
receive the encoded data set from the one of a plurality of implantable medical device types via a communication network;
identify the one of the plurality of medical device types; and
direct the encoded data set to the one of the plurality of ports corresponding to the one of the plurality of implantable medical device types.

19. The system of claim 14, wherein the computer readable medium further includes instructions executable by the processor to:
store the abstracted data set to a storage area selected from a group consisting of: a comprehensive database, and a subset database.

20. The system of claim 11, wherein the computer readable medium further includes instructions executable by the processor to:
translate the abstracted data set to a selected format data set.

21. The system of claim 20, wherein the processor is a first processor, and wherein the computer readable medium is a first computer readable medium, wherein the system further comprises a system server, wherein the system server includes a second processor and a second computer readable medium, and wherein the second computer readable medium includes instructions executable by the processor to:
receive the selected format data set; and
communicate the selected format data set to a recipient.

22. A method for utilizing information from implantable medical devices, the method comprising:
providing a first communication link;
providing a first conversion utility associated with the first communication link;
providing a second communication link;
providing a second conversion utility associated with the second communication link;
assigning a first group of medical devices to the first communication link;
assigning a second group of medical devices to the second communication link;
receiving a first data set from a first implantable medical device from the first group of medical devices;
communicating the first data set to the first conversion utility via the first communication link, wherein a converted data set is created; and
receiving the converted data set.

23. The method of claim 22, wherein the first communication link includes a first server port, and wherein the second communication link comprises a second server port.

24. The method of claim 22, wherein the method further comprises:
receiving the first data set via the first communication link;
decoding the first data set to create a decoded data set; and
abstracting the first data set to create the converted data set.

25. The method of claim 22, wherein the converted data set is an standardized format data set.

26. The method of claim 22, wherein the method further comprises:
identifying the first data set as originating from an implantable medical device included within the first group of implantable medical devices.

27. The method of claim 22, wherein the converted data set is a first converted data set, and wherein the method further comprises:
receiving a second data set from a second implantable medical device from the second group of medical devices;
communicating the second data set to the second conversion utility via the second communication link, wherein a second converted data set is created; and
receiving the second converted data set.

28. The method of claim 27, the method further comprising:
storing the first converted data set and the second converted data set to a comprehensive database.

29. The method of claim 27, the method further comprising:
distributing at least a first portion of the first converted data set and the second converted data set to a first recipient; and
distributing at least a second portion of the first converted data set and the second converted data set to a second recipient.
